(19) United States
(12) Patent Application Publication
(10) Pub. No.: US 2012/0237590 A1
(43) Pub. Date: Sep. 20, 2012

(54) CURCUMIN COMBINATION WITH ANTI-TYPE 2 DIABETIC DRUGS FOR PREVENTION AND TREATMENT OF DISEASE SEQUELAE, DRUG-RELATED ADVERSE REACTIONS, AND IMPROVED GLYCEMIC CONTROL

(75) Inventor: Lawrence Helson, Quakertown, PA (US)

(73) Assignee: SIGNPATH PHARMA INC., Quakertown, PA (US)

(21) Appl. No.: 13/421,221

(22) Filed: Mar. 15, 2012

Related U.S. Application Data
(60) Provisional application No. 61/453,359, filed on Mar. 16, 2011.

(51) Int. Cl. A61K 38/28; A61K 31/155; A61K 38/22; A61K 9/14; A61P 3/10; A61P 25/00; A61P 27/02; A61P 13/12; A61P 1/18; A61P 35/00; A61P 9/00; A61P 25/02; A61K 31/12; A61K 31/56; A61K 31/4184; A61K 38/18; A61K 31/554; A61K 31/438; A61K 31/137; A61K 9/127

(52) U.S. Cl. 424/450; 514/5.9; 514/635; 514/6.9; 424/497; 514/679; 514/171; 514/394; 514/7.7; 514/211.07; 514/278; 514/649

(57) ABSTRACT

Compositions and methods for treating type 2 diabetes and its sequelae by intravenous or subcutaneous administration of formulations of synthesized curcumin (diferuloylmethane) and concomitantly one or more anti-diabetic agents to human subjects are disclosed herein. The composition of the present invention may be used to: (i) treat patients with diabetes in advanced stages with evidence of any or all encephalopathy, retinopathy, nephropathy, pancreatitis or neoplasias; (ii) treat patients with diabetic disease status without symptomatic or pathologic evidence of associated sequelae but requiring better glycemic control than that offered by standard of care anti-diabetic; and (iii) patients with objective signs or symptoms of sequelae from diabetes of anti-diabetic drugs. One three-drug combination of the present invention includes a slow release PLGA-curcumin and an oral gliptin (DPP-4)-inhibitor or any incretin-mimetic and metformin.

### CROSS-REFERENCE TO RELATED APPLICATIONS

This application claims priority to and benefit of U.S. Provisional Application Ser. No. 61/453,359, filed on Mar. 16, 2011, entitled: Curcumin combination with anti-type 2 diabetic drugs for prevention and treatment of disease sequelae, drug-related adverse reactions, and improved glycemic control, by inventor Lawrence Helson [Attorney Docket No. SPPI:1004P].

### TECHNICAL FIELD OF THE INVENTION

The present invention relates in general to the field of metabolic (diabetes), neoplastic (cancer), nephropathy, and neuro-vascular-degenerative diseases (encephalopathies, retinopathies), and more particularly to the intravenous or subcutaneous administration of formulations of synthesized curcumin (diferuloylmethane) and concomitantly oral or injectable or both anti-diabetic drugs to human subjects with type 2 diabetes in need of treatment against metabolic, renal, retinal neurodegenerative, and neoplastic diseases.

### STATEMENT OF FEDERALLY FUNDED RESEARCH

None.

### REFERENCE TO A SEQUENCE LISTING

None.

### BACKGROUND OF THE INVENTION

Epidemiologic data suggests that uncontrolled diabetes (hyperglycemia) is a predisposing and key initiating factor for acute and chronic morbidity including pancreatitis, encephalopathy, retinopathy, autonomic peripheral neuropathies, nephropathy and neoplastic diseases.

Without limiting the scope of the invention, its background is described in connection with the use and dosage forms of anti-diabetic chemotherapeutic agents, and agents for treating neoplastic (cancer), neurodegenerative, metabolic diseases (type 2 diabetes and its encephalopathic, retinal, renal and neoplastic sequelae) when combined with curcumin formulations. The drugs are characterized as insulin, sulfonylurea secretagogues, non-sulfonylurea secretagogues (Meglitinides), sensitizers (Biguanides, Thiozolidinedions), Alpha-glucosidase inhibitors, Peptide analogs (incretin-mimetics, glucagon-like peptide analogs and agonists, gastric inhibitory peptide analogs, injectable peptide analogs) and Amylin analogues.

U.S. Patent Application Publication No. 20100239552 (Mayoux et al. 2010) is directed to pharmaceutical combinations comprising an antioxidant agent, an anti-inflammatory agent, and optionally at least one other anti-diabetic agent useful for treating metabolic disorders. This invention also encompasses pharmaceutically acceptable compositions comprising an antioxidant agent, an anti-inflammatory agent, optionally at least one other anti-diabetic agent, and at least one pharmaceutically acceptable carrier. The combinations and compositions of this invention are useful as methods for treating metabolic disorders including diabetes, particularly Type I and Type II diabetes, as well as diseases and disorders associated with diabetes, including but not limited to atherosclerosis, cardiovascular disease, inflammatory disorders, nephropathy, neuropathy, retinopathy, β-cell dysfunction, dyslipidemia, LADA, metabolic syndrome, hyperglycemia, insulin resistance, and/or chronic obstructive pulmonary disease in a mammal, particularly a diabetic mammal, and specifically a human patient. The antioxidant agent in the Mayoux invention comprises resveratrol, silibinin, alpha-lipoic acid or a pharmaceutically acceptable salt thereof, pterostilbene, N-acetyl cysteine, taurine, probucol, idebenone or curcumin.

WIPO Patent Application Publication No. WO/2004/047717 (Elmallah et al. 2004) discloses topical application of Curcumin (from Curcuma longa, Turmeric) in treating peripheral neuropathies, including diabetic neuropathy associated with chronic type I or Type II diabetes mellitus. Concentrations of 0.025-2% Curcumin were effective. The topical application in the form of a cream, an ointment, a gel or a solution.

U.S. Patent Application Publication No. 20100240581 (Tortoriello and Weisberg, 2010) discloses methods of modulating chronic low-grade inflammation are provided. More particularly, methods of treating diabetes, such as for example, type-2 diabetes mellitus, in a mammal by administering an effective amount of a selective proteasome inhibitor are provided. Also provided are unit dosage forms of such inhibitors. The selective proteasome inhibitor in the Tortoriello invention is selected from the group consisting of curcumin, epoxomicin, celastrol, derivatives thereof, and combinations thereof.

U.S. Patent Application Publication No. 20100179103 (Desai, 2010) discloses the anti-inflammatory and anti-angiogenic properties of Curcumin that could be useful in treating various diseases such as those of rheumatology and oncology. However, curcumin is very poorly absorbed and has a very low bioavailability. The Desai invention describes a method of increasing the delivery of curcumin by complexing it with cyclodextrins. Cyclodextrins are well known in the food industry and have been used to carry other drugs to increase bioavailability. The new combination of cyclodextrins and curcumin has been tested in pre-clinical inflammation models where it has demonstrated efficacy superior to both the positive control and curcumin. The diseases that can be treated by the composition in the Desai invention include but are not limited to neurological diseases such as Alzheimers, autoimmune or inflammatory or allergic diseases such as asthma and rheumatoid arthritis, oncologic diseases, respiratory system diseases such as chronic obstructive lung disease, dermatologic diseases, cardiovascular diseases such as hyperlipidemia and coronary artery disease, gastrointestinal hepatic and pancreatic diseases such as inflammatory bowel disease, metabolic diseases such as diabetes, urologic, infectious diseases and for wound healing.

### SUMMARY OF THE INVENTION

The present invention describes the intravenous or subcutaneous administration of formulations of synthesized curcumin (diferuloylmethane) and concomitantly oral or injectable or both anti-diabetic drugs to human subjects, with type 2 diabetes, in need of treatment, against metabolic, renal, retinal neurodegenerative and neoplastic diseases. Particular reference is made to the repeated intravenous administration of liposomal curcumin, or polymeric nanocurcumin or to the sustained release of curcumin from PLGA nanocurcumin at dosages below hemolytic thresholds concomitantly with oral or parenteral anti-diabetic drugs. Reference is also made to the prevention or lowering of the incidence or risk of retinopathy, nephropathy, pancreatitis, pancreatic, thyroid and other cancers in patients receiving combination therapy with curcumin formulations. There are four formulations: curcumin encapsulated in a spherical liposome, or enclosed within a polymeric nanoparticle, or conjugated to one or more biodegradable polymers, or a curcumin encapsulated in a liposome and conjugated to one or more biodegradable polymers. In one aspect, the present invention includes a lipid nanosphere that is formulated to deliver curcumin directly into the circulation via the intravenous route. Once in the circulation the lipid based nanoparticulates provide delivery to systemic tissues and their cellular contents for example circulating, hepatic, splenic and bone marrow macrophages. The liposomal curcumin made up of DMPC DPMG is stable in blood, plasma, and culture medium. Curcumin enclosed in a polymeric nanoparticle composed of at least one of N-isopropylacrylamide, (NIPAAM), N-vinyl pyrrolidinone (VP) and acrylic acid (AA), is a composition capable of solubilizing a broad range of poorly water-soluble drugs. Curcumin is fully soluble in this formulation and can be used for injection. For example, this formulation upon daily intraperitoneal injection in mice exhibits high bioavailibility and is non-toxic. Yet another embodiment is a nanoparticulate formulation of curcumin amenable to systemic administration. Another formulation includes curcumin in PLGA nanoshell composed of poly-lactic acid, poly-glycolic acid, poly-lactic-co glycolic acid and combinations thereof, which allows controlled intravascular sustained release and when combined with a targeting agent it effectively controls the size and drug delivery rate to diseased tissue/cells minimizing whole body dose. The cardiac potassium channel blocking activity of curcumin is abrogated in the liposomal and the nanoparticle formulations, or as unfettered liposomes or polymers. While the PLGA-curcumin does not exhibit this protective activity, another formulation was developed fourth that is a hybridized formulation that includes liposomal curcumin surrounded by PLGA. The PLGA surrounded curcumin liposomes avoid the potential cardiotoxicity associated with PLGA curcumin.

In one embodiment, the present invention includes a composition for ameliorating symptoms and/or treating type 2 diabetes and one or more associated pathological conditions (sequelae) in a human subject comprising: a therapeutically effective amount of a formulation comprising curcumin, curcumin analogues, curcumin derivatives, curcuminoids or combinations thereof dissolved or dispersed in a suitable aqueous or non-aqueous medium, wherein the curcumin is enclosed in at least one of one or more liposomes, enclosed in a polymeric nanoparticle, conjugated to one or more biodegradable polymers or formulated into a liposome that is surrounded or incorporated into a polymeric nanoparticle; a therapeutically effective amount of one or more anti-diabetic agents; and one or more optional excipients, diluents, extended or controlled release agents, lubricants, preservatives or any combinations thereof. In one aspect, the formulation is administered concomitantly with the one or more anti-diabetic agents, wherein the anti-diabetic agents may be administered orally, intravenously, or subcutaneously. In one aspect, the type 2 diabetes associated pathological condition is at least one of cerebral, cardiac, pancreatic, renal, an ocular condition, pancreatitis, retinopathy, cardiopathy, encephalopathy, peripheral neuropathy and renopathy, or increased cancer incidence. In one aspect, the one or more biodegradable polymers are selected from the group consisting of polyesters, polylactides, polyglycolides, polycaprolactones, polyanhydrides, polyamides, polyurethanes, polyesteramides, polydioxanones, polyacetals, polyketals, polycarbonates, polyorthocarbonates, polyorthoesters, polyphosphoesters, polyphosphazenes, polyhydroxybutyrates, polyhydroxyvalerates, polyalkylene oxalates, polyalkylene succinates, poly(malic acid), poly(amino acids), copolymers, poly-lactic glycolic acid (PLGA) copolymer, terpolymers, and combinations or mixtures thereof. In one aspect, the PLGA copolymer enclosed curcumin releases 50% of the curcumin within the first 24 hours and 20-50% of the remaining curcumin within 2-10 days following a subcutaneous injection.

In one aspect, the liposome comprises a lipid or a phospholipid wall, wherein the lipids or the phospholipids are selected from the group consisting of phosphatidylcholine (lecithin), lysolecithin, lysophosphatidylethanol-amine, phosphatidylserine, phosphatidylinositol, sphingomyelin, phosphatidylethanolamine (cephalin), cardiolipin, phosphatidic acid, cerebrosides, dicetylphosphate, phosphatidylcholine, and dipalmitoyl-phosphatidylglycerol, stearylamine, dodecylamine, hexadecyl-amine, acetyl palmitate, glycerol ricinoleate, hexadecyl sterate, isopropyl myristate, amphoteric acrylic polymers, fatty acid, fatty acid amides, cholesterol, cholesterol ester, diacylglycerol, and diacylglycerolsuccinate. In one aspect, the anti-diabetic agent comprises insulin, pramlitide, bydureone, metformin, oral incretin mimetics, metformin, or any combinations thereof. In one aspect, the formulation may be administered with one or more agents to prevent hemolysis, hypersensitivity reactions or both, wherein the agents comprise calcium channel blockers, antihistamines, corticosteroid or any combinations thereof. In one aspect, the calcium channel blockers comprise verapamil, ethylisopropylameloride, niflamic acid, NPPB, dihydropyridines, phenylalkylamines, benzothiozepines, diltiazem, non-selective blockers comprising mibefradil, bepredil, fendeline, fluspirilene, catecholamines, and erythropoietin agents. In one aspect, the nanoparticle comprises a copolymer comprising isopropylacrylamide, (NIPAAM), N-vinyl pyrrolidinone (VP) and acrylic acid (AA).

Another embodiment of the present invention includes a method for ameliorating symptoms and/or treating type 2 diabetes and one or more associated pathological conditions (sequelae) in a human subject comprising the steps of: identifying the human subject in need of amelioration of the symptoms and/or treatment of type 2 diabetes and one or more associated pathological conditions (sequelae); and administering a therapeutically effective amount of a pharmaceutical composition to the human subject, wherein the composition comprises: curcumin, curcumin analogues, curcumin derivatives, curcuminoids or combinations thereof dissolved or dispersed in a suitable aqueous or non-aqueous medium, wherein the curcumin is enclosed in at least one of one or more liposomes, enclosed in a polymeric nanoparticle, conjugated to one or more biodegradable polymers or formulated into a liposome that is surrounded or incorporated into a polymeric nanoparticle; one or more anti-diabetic agents; and one or more optional excipients, diluents, extended or controlled release agents, lubricants, preservatives or any combinations thereof. In one aspect, the formulation is administered concomitantly with the one or more anti-diabetic agents, wherein the anti-diabetic agents may be administered orally, intravenously, or subcutaneously. In one aspect, the type 2 diabetes associated pathological condition is a cerebral, cardiac, pancreatic, renal, or an ocular condition pancreatitis, retinopathy, cardiopathy, encephalopathy, peripheral neuropathy and renopathy, increased cancer incidence, or any combinations thereof. In one aspect, the one or more biodegradable polymers are selected from the group consisting of polyesters, polylactides, polyglycolides, polycaprolactones, polyanhydrides, polyamides, polyurethanes, polyesteramides, polydioxanones, polyacetals, polyketals, polycarbonates, polyorthocarbonates, polyorthoesters, polyphosphoesters, polyphosphazenes, polyhydroxybutyrates, polyhydroxyvalerates, polyalkylene oxalates, polyalkylene succinates, poly(malic acid), poly(amino acids), copolymers, poly-lactic glycolic acid (PLGA) copolymer, terpolymers, and combinations or mixtures thereof.

In one aspect, the liposome comprises a lipid or a phospholipid wall, wherein the lipids or the phospholipids are selected from the group consisting of phosphatidylcholine (lecithin), lysolecithin, lysophosphatidylethanol-amine, phosphatidylserine, phosphatidylinositol, sphingomyelin, phosphatidylethanolamine (cephalin), cardiolipin, phosphatidic acid, cerebrosides, dicetylphosphate, phosphatidylcholine, and dipalmitoyl-phosphatidylglycerol, stearylamine, dodecylamine, hexadecyl-amine, acetyl palmitate, glycerol ricinoleate, hexadecyl sterate, isopropyl myristate, amphoteric acrylic polymers, fatty acid, fatty acid amides, cholesterol, cholesterol ester, diacylglycerol, and diacylglycerolsuccinate. In one aspect, the PLGA copolymer enclosed curcumin releases 50% of the curcumin within the first 24 hours and 20-50% of the remaining curcumin within 2-10 days following a subcutaneous injection. In one aspect, the anti-diabetic agent comprises insulin, pramlitide, bydureone, metformin, oral incretin mimetics, metformin, or any combinations thereof. In one aspect, the formulation may be administered with one or more agents to prevent hemolysis, hypersensitivity reactions or both, wherein the agents comprise calcium channel blockers, antihistamines, corticosteroid or any combinations thereof. In one aspect, the calcium channel blockers comprise verapamil, ethylisopropylameloride, niflamic acid, NPPB, dihydropyridines, phenylalkylamines, benzothiozepines, diltiazem, non-selective blockers comprising mibefradil, bepredil, fendeline, fluspirilene, catecholamines, and erythropoietin agents.

In another embodiment the present invention includes a pharmaceutical composition for combination therapy to provide enhanced glycemic control in type 2 diabetes, prevent or treat one or more pathological conditions associated with type 2 diabetes, or both comprising: curcumin, curcumin analogues, curcumin derivatives, curcuminoids or combinations thereof dissolved or dispersed in a suitable aqueous or non-aqueous medium, wherein the curcumin is enclosed in at least one of one or more liposomes, enclosed in a polymeric nanoparticle, conjugated to one or more biodegradable polymers or formulated into a liposome that is surrounded or incorporated into a polymeric nanoparticle; and one or more anti-diabetic agents comprising DPP-4 inhibitors, insulin release sensitizers, glucagon modifiers or any combinations thereof. In one aspect, the composition comprises Curcumin-DPP4-inhibitor-metformin. In one aspect, the composition comprises Curcumin-bydureon-slow release-metformin. In one aspect, the composition has fewer adverse reactions than DPP4-inhibitor-metformin, bydureon-slow release-metformin or both. In one aspect, the composition provides enhanced glycemic control when compared to DPP4-inhibitor-metformin, bydureon-slow release-metformin or both.

In another embodiment the present invention includes a method of providing enhanced glycemic control in type 2 diabetes, preventing or treating one or more pathological conditions associated with type 2 diabetes, or both in a human subject comprising the steps of: identifying the human subject in need of enhanced glycemic control in type 2 diabetes, prevention or treatment of one or more pathological conditions associated with type 2 diabetes, or both; and administering a therapeutically effective amount of a pharmaceutical composition to the human subject comprising: curcumin, curcumin analogues, curcumin derivatives, curcuminoids or combinations thereof dissolved or dispersed in a suitable aqueous or non-aqueous medium, wherein the curcumin is enclosed in at least one of one or more liposomes, enclosed in a polymeric nanoparticle, conjugated to one or more biodegradable polymers or formulated into a liposome that is surrounded or incorporated into a polymeric nanoparticle; and one or more anti-diabetic agents comprising DPP-4 inhibitors, insulin release sensitizers, glucagon modifiers or any combinations thereof. In one aspect, the one or more pathological conditions associated with type 2 diabetes comprise encephalopathy, retinopathy, nephropathy, pancreatitis, pancreatic, cancer thyroid cancer, and other cancers. In one aspect, the composition may comprise one or more excipients, diluents, extended or controlled release agents, lubricants, preservatives or any combinations thereof. In one aspect, the composition comprises Curcumin-DPP4-inhibitor-metformin or Curcumin-bydureon-slow release-metformin.

In another embodiment the present invention includes a pharmaceutical composition for combination therapy to provide enhanced glycemic control in type 2 diabetes, prevent or treat one or more pathological conditions associated with type 2 diabetes, ameliorate one or more adverse reactions associated with type 2 diabetes treatment or any combinations thereof comprising: curcumin, curcumin analogues, curcumin derivatives, curcuminoids or combinations thereof dissolved or dispersed in a suitable aqueous or non-aqueous medium, wherein the curcumin is enclosed in at least one of one or more liposomes, enclosed in a polymeric nanoparticle, conjugated to one or more biodegradable polymers or formulated into a liposome that is surrounded or incorporated into a polymeric nanoparticle; and one or more anti-diabetic agents comprising DPP-4 inhibitors, insulin release sensitizers, glucagon modifiers or any combinations thereof.

In another embodiment the present invention includes a method of providing enhanced glycemic control in type 2 diabetes, preventing or treating one or more pathological conditions associated with type 2 diabetes, ameliorating adverse reactions associated with type 2 diabetes treatment or any combinations thereof in a human subject comprising the steps of: identifying the human subject in need of enhanced glycemic control in type 2 diabetes, prevention or treatment of one or more pathological conditions associated with type 2 diabetes, amelioration or prevention of adverse reactions associated with type 2 diabetes treatment or any combinations thereof; and administering a therapeutically effective amount of a pharmaceutical composition to the human subject comprising: curcumin, curcumin analogues, curcumin derivatives, curcuminoids or combinations thereof dissolved or dispersed in a suitable aqueous or non-aqueous medium, wherein the curcumin is enclosed in at least one of one or more liposomes, enclosed in a polymeric nanoparticle, conjugated to one or more biodegradable polymers or formulated into a liposome that is surrounded or incorporated into a polymeric nanoparticle; and one or more anti-diabetic agents comprising DPP-4 inhibitors, insulin release sensitizers, glucagon modifiers or any combinations thereof.

In another embodiment the present invention includes a composition for ameliorating symptoms and/or treating type 2 diabetes and one or more associated pathological conditions (sequelae) in a human subject comprising: a therapeutically effective amount of a formulation comprising curcumin, curcumin analogues, curcumin derivatives, curcuminoids or combinations thereof enclosed in a liposome, and wherein the liposomes are enclosed in a polymeric nanoparticle, wherein the formulation inhibits a cardiac potassium channel blocking activity of curcumin; and one or more optional excipients, diluents, extended or controlled release agents, lubricants, preservatives or any combinations thereof. In one aspect, the formulation is administered concomitantly with the one or more anti-diabetic agents, wherein the anti-diabetic agents may be administered orally, intravenously, or subcutaneously. In one aspect, the type 2 diabetes associated pathological condition is at least one of cerebral, cardiac, pancreatic, renal, an ocular condition, pancreatitis, retinopathy, cardiopathy, encephalopathy, peripheral neuropathy and renopathy, or increased cancer incidence. In one aspect, the one or more biodegradable polymers are selected from the group consisting of polyesters, polylactides, polyglycolides, polycaprolactones, polyanhydrides, polyamides, polyurethanes, polyesteramides, polydioxanones, polyacetals, polyketals, polycarbonates, polyorthocarbonates, polyorthoesters, polyphosphoesters, polyphosphazenes, polyhydroxybutyrates, polyhydroxyvalerates, polyalkylene oxalates, polyalkylene succinates, poly(malic acid), poly(amino acids), copolymers, poly-lactic glycolic acid (PLGA) copolymer, terpolymers, and combinations or mixtures thereof. In one aspect, the PLGA copolymer enclosed curcumin releases 50% of the curcumin within the first 24 hours and 20-50% of the remaining curcumin within 2-10 days following a subcutaneous injection.

In one aspect, the liposome comprises a lipid or a phospholipid wall, wherein the lipids or the phospholipids are selected from the group consisting of phosphatidylcholine (lecithin), lysolecithin, lysophosphatidylethanol-amine, phosphatidylserine, phosphatidylinositol, sphingomyelin, phosphatidylethanolamine (cephalin), cardiolipin, phosphatidic acid, cerebrosides, dicetylphosphate, phosphatidylcholine, and dipalmitoyl-phosphatidylglycerol, stearylamine, dodecylamine, hexadecyl-amine, acetyl palmitate, glycerol ricinoleate, hexadecyl sterate, isopropyl myristate, amphoteric acrylic polymers, fatty acid, fatty acid amides, cholesterol, cholesterol ester, diacylglycerol, and diacylglycerolsuccinate. In one aspect, the anti-diabetic agent comprises insulin, pramlitide, bydureone, metformin, oral incretin mimetics, metformin, or any combinations thereof. In one aspect, the formulation may be administered with one or more agents to prevent hemolysis, hypersensitivity reactions or both, wherein the agents comprise calcium channel blockers, antihistamines, corticosteroid or any combinations thereof. In one aspect, the calcium channel blockers comprise verapamil, ethylisopropylameloride, niflamic acid, NPPB, dihydropyridines, phenylalkylamines, benzothiozepines, diltiazem, non-selective blockers comprising mibefradil, bepredil, fendeline, fluspirilene, catecholamines, and erythropoietin agents. In one aspect, the nanoparticle comprises a copolymer comprising isopropylacrylamide, (NIPAAM), N-vinyl pyrrolidinone (VP) and acrylic acid (AA).

In another embodiment the present invention includes a method of providing enhanced glycemic control in type 2 diabetes, preventing or treating one or more pathological conditions associated with type 2 diabetes, ameliorating adverse reactions associated with type 2 diabetes treatment or any combinations thereof in a human subject comprising the steps of: identifying the human subject in need of enhanced glycemic control in type 2 diabetes, prevention or treatment of one or more pathological conditions associated with type 2 diabetes, amelioration or prevention of adverse reactions associated with type 2 diabetes treatment or any combinations thereof; and administering a therapeutically effective amount of a pharmaceutical composition to the human subject comprising: a therapeutically effective amount of a formulation comprising curcumin, curcumin analogues, curcumin derivatives, curcuminoids or combinations thereof enclosed in a liposome, and wherein the liposomes are enclosed in a polymeric nanoparticle, wherein the formulation inhibits a cardiac potassium channel blocking activity of curcumin; and one or more optional excipients, diluents, extended or controlled release agents, lubricants, preservatives or any combinations thereof. In one aspect, the formulation is administered concomitantly with the one or more anti-diabetic agents, wherein the anti-diabetic agents may be administered orally, intravenously, or subcutaneously. In one aspect, the type 2 diabetes associated pathological condition is at least one of cerebral, cardiac, pancreatic, renal, an ocular condition, pancreatitis, retinopathy, cardiopathy, encephalopathy, peripheral neuropathy and renopathy, or increased cancer incidence. In one aspect, the one or more biodegradable polymers are selected from the group consisting of polyesters, polylactides, polyglycolides, polycaprolactones, polyanhydrides, polyamides, polyurethanes, polyesteramides, polydioxanones, polyacetals, polyketals, polycarbonates, polyorthocarbonates, polyorthoesters, polyphosphoesters, polyphosphazenes, polyhydroxybutyrates, polyhydroxyvalerates, polyalkylene oxalates, polyalkylene succinates, poly(malic acid), poly(amino acids), copolymers, poly-lactic glycolic acid (PLGA) copolymer, terpolymers, and combinations or mixtures thereof. In one aspect, the PLGA copolymer enclosed curcumin releases 50% of the curcumin within the first 24 hours and 20-50% of the remaining curcumin within 2-10 days following a subcutaneous injection.

In one aspect, the liposome comprises a lipid or a phospholipid wall, wherein the lipids or the phospholipids are selected from the group consisting of phosphatidylcholine (lecithin), lysolecithin, lysophosphatidylethanol-amine, phosphatidylserine, phosphatidylinositol, sphingomyelin, phosphatidylethanolamine (cephalin), cardiolipin, phosphatidic acid, cerebrosides, dicetylphosphate, phosphatidylcholine, and dipalmitoyl-phosphatidylglycerol, stearylamine, dodecylamine, hexadecyl-amine, acetyl palmitate, glycerol ricinoleate, hexadecyl sterate, isopropyl myristate, amphoteric acrylic polymers, fatty acid, fatty acid amides, cholesterol, cholesterol ester, diacylglycerol, and diacylglycerolsuccinate. In one aspect, the anti-diabetic agent comprises insulin, pramlitide, bydureone, metformin, oral incretin mimetics, metformin, or any combinations thereof. In one aspect, the formulation may be administered with one or more agents to prevent hemolysis, hypersensitivity reactions or both, wherein the agents comprise calcium channel blockers, antihistamines, corticosteroid or any combinations thereof. In one aspect, the calcium channel blockers comprise verapamil, ethylisopropylameloride, niflamic acid, NPPB, dihydropyridines, phenylalkylamines, benzothiozepines, diltiazem, non-selective blockers comprising mibefradil, bepredil, fendeline, fluspirilene, catecholamines, and erythropoietin agents. In one aspect, the nanoparticle comprises a copolymer comprising isopropylacrylamide, (NIPAAM), N-vinyl pyrrolidinone (VP) and acrylic acid (AA).

### BRIEF DESCRIPTION OF THE DRAWINGS

For a more complete understanding of the features and advantages of the present invention, reference is now made to the detailed description of the invention along with the accompanying figures and in which:

FIG. 1 is an in vitro sustained release profile of a formulation comprising PLGA-CURC nanoparticles. The formulation was prepared using a blend of two polymers 50% of PLGA (50/50) and 50% of PLGA (85/15). The study was performed in triplicate from the same batch of nanoparticle formulation.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “Curcumin (diferuloyl methane; 1,7-bis(4-hydroxy-3-methoxyphenyl)-1,6-heptadiene-3,5-dione)” is a naturally occurring compound which is the main coloring principle found in the rhizomes of the plant Curcuma longa (U.S. Pat. No. 5,679,864, Krackov et al.).

As used herein, the term “liposome” refers to a capsule wherein the wall or membrane thereof is formed of lipids, especially phospholipid, with the optional addition therewith of a sterol, especially cholesterol.

As used herein, the term “in vivo” refers to being inside the body. The term “in vitro” used as used in the present application is to be understood as indicating an operation carried out in a non-living system.

As used herein, the term “receptor” includes, for example, molecules that reside on the surface of cells and mediate activation of the cells by activating ligands, but also is used generically to mean any molecule that binds specifically to a counterpart. One member of a specific binding pair would arbitrarily be called a “receptor” and the other a “ligand.” No particular physiological function need be associated with this specific binding. Thus, for example, a “receptor” might include antibodies, immunologically reactive portions of antibodies, molecules that are designed to complement other molecules, and so forth. Indeed, in the context of the present invention, the distinction between “receptor” and “ligand” is entirely irrelevant; the invention concerns pairs of molecules that specifically bind each other with greater affinity than either binds other molecules. However, for ease of explanation, the invention method will be discussed in terms of target receptor (again, simply a molecule for which a counterpart is sought that will react or bind with it) and “ligand” simply represents that counterpart.

As used herein, the term “treatment” refers to the treatment of the conditions mentioned herein, particularly in a patient who demonstrates symptoms of the disease or disorder.

As used herein, the term “treatment” or “treating” refers to any administration of a compound of the present invention and includes: (i) inhibiting the disease in an animal that is experiencing or displaying the pathology or symptomatology of the diseased (i.e., arresting further development of the pathology and/or symptomatology); or (ii) ameliorating the disease in an animal that is experiencing or displaying the pathology or symptomatology of the diseased (i.e., reversing the pathology and/or symptomatology). The term “controlling” includes preventing treating, eradicating, ameliorating or otherwise reducing the severity of the condition being controlled.

As used herein, the terms “effective amount” or “therapeutically effective amount” described herein means the amount of the subject compound that will elicit the biological or medical response of a tissue, system, animal, or human that is being sought by the researcher, veterinarian, medical doctor or other clinician.

As used herein, the terms “administration of” or “administering a” compound as used herein should be understood to mean providing a compound of the invention to the individual in need of treatment in a form that can be introduced into that individual's body in a therapeutically useful form and therapeutically useful amount, including, but not limited to: oral dosage forms, such as tablets, capsules, syrups, suspensions, and the like; injectable dosage forms, such as IV, IM, or IP, and the like; transdermal dosage forms, including creams, jellies, powders, or patches; buccal dosage forms; inhalation powders, sprays, suspensions, and the like; and rectal suppositories.

As used herein, the term “intravenous administration” includes injection and other modes of intravenous administration.

As used herein, the term “pharmaceutically acceptable” as used herein to describe a carrier, diluent or excipient must be compatible with the other ingredients of the formulation and not deleterious to the recipient thereof.

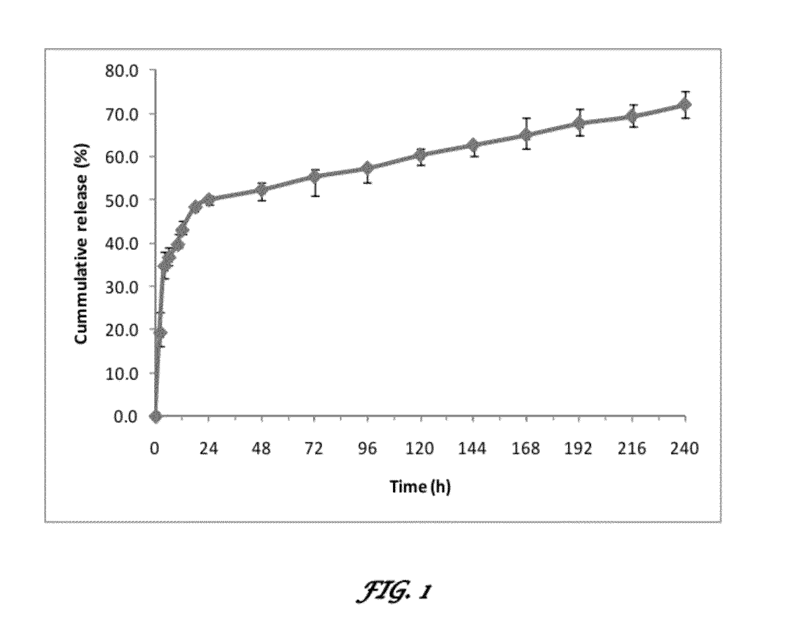

The present invention includes compositions and methods for treating type 2 diabetes and its sequelae. The present invention discloses a formulated pharmaceutical composition comprising a therapeutically effective amount of a formulated synthesized curcumin (diferuloylmethane) wherein the synthesized curcumin is enveloped by a poly-lactic glycolic acid (PLGA) copolymer with the characteristic of a rapid release of 50% of a subcutaneous injected bolus within the first 24 hours, followed by a slow release over days 2-10 of the remaining 20-50% (FIG. 1).

In patients with diabetes in advanced stages with evidence of any or all encephalopathy, retinopathy, nephropathy, pancreatitis or neoplasias may be treated by administration of slow-release PLGA-curcumin, liposomal curcumin, or polymeric nanocurcumin concomitantly with any subcutaneous, intravenous, or orally administered anti-diabetic drugs. For patients with diabetic disease status without symptomatic or pathologic evidence of associated sequelae but requiring better glycemic control than that offered by standard of care anti-diabetic drugs (oral or subcutaneously administered drugs, PLGA-curcumin a sustained release curcumin formulation at doses below systemic hemolytic thresholds may be added to conventional treatment. For patients with objective signs or symptoms of sequelae from diabetes of anti-diabetic drugs, then a combination of quick release liposomal curcumin, or nanocurcumin formulations followed by slow release curcumin formulation may be added to conventional treatment.

The preferred three drug combinations are slow release PLGA-curcumin and an oral gliptin (DPP-4)-inhibitor or any incretin-mimetic and metformin. For practical applications combined subcutaneous injection of any of these curcumin formulations with Byetta, or the long acting bydureon, or Victoza and metformin may prove efficacious for diabetic glycemic control.

The formulations described in the present invention are constructed of a layer of lipids to form a liposome or a layer of acrylic acid polymers may also be used. The latter may require different scheduling depending upon concomitant anti-diabetic drugs being used and severity of the disease. The PLGA curcumin formulations may be co-injected subcutaneously with insulin, pramlitide or bydureone (Amylin Inc. and Lilly, Inc.) and concomitantly with oral drugs such as metformin. They may also be injected subcutaneously once weekly along with oral incretin-mimetics (example: Sitagluptin, Saxagliptin) and metformin. The compositions of the present invention are adapted for intravenous or a subcutaneous administration in a human subject for treatment of type-2 diabetes and its many pathologic cerebral, cardiac, pancreatic, ocular and renal sequelea related to the diabetic state, or exacerbated by treatment oral or injectable anti-diabetic drugs.

As stated hereinabove the curcumin formulation is administered prior to or concomitantly with the other diabetic drugs. The curcumin formulation of the present invention may comprise one or more optional pharmaceutical excipients, diluents, extended or controlled release agents, lubricants, preservatives or any combination thereof, and once solubilized may be added to injectable anti-diabetic medications or administered in a schedule depending upon the release kinetics of the curcumin formulation. A large number of biodegradable polymers may be used in the formulation of the present invention. Non-limiting examples of these polymers include polysesters, polylactides, polyglycolides, polycaprolactones polyanhydrides, polyamides, polyurethanes, polyesteramides, polydiaxanones, polyacetals, polyketals, polycarbonates, polyorthocarbonates, polyorthoesters, polyphosphoesters, polyphosphazenes, polyhydroxybuterates, polyhydroxyvalerates, polyalkelene oxalates, polyalkylene succinates, poly(malic)acid, poly(amino)acids, copolymers, terpolymers, and combinations or mixtures thereof. Specific polymers that may be used include an acrylic acid, a vinylpyrrolidinome, a N-isopropylacrylamide or combinations and modifications thereof. The synthesized curcumin that is used includes curcumin, curcumin analogues, curcumin derivatives and any modifications thereof.

The present invention further provides a method comprising the subcutaneous or intravenous administration to a subject of a therapeutically effective amount of a formulated composition of synthesized curcumin (diferuloylmethane) wherein the synthesized curcumin is enveloped by a polylactic glycolic acid (PLGA) copolymer with a specific ratio permitting continuous pulsed release the first 24 hours and continued release over the next nine days, a layer of lipids to form a liposome conjugated to one or more polymers or any combination thereof in combination with one or more calcium channel blockers to mitigate intravenous liposomal induce red blood cell hemolysis. The calcium channel blockers are selected from the group consisting of verapamil, ethylisopropylameloride, niflamic acid, NPPB, dihydropyridines, phenylalkylamines, benzothiozepines, diltiazem, non-selective blockers comprising mibefradil, bepredil,fendeline, fluspirilene, catecholamines, and erythropoietin agents. A polymer used in the present invention is an acrylic acid, a vinylpyrrolidinome, a N-isopropylacrylamide or combinations or modifications thereof.

Furthermore a method of treating a subject afflicted with type-2 diabetes is also disclosed herein. The method comprises the steps of: (i) identifying the subject in need of treatment against diabetic morbidity, the sequelae associated with the disease and the sequelae associated with the various treatment methods, such as increased cancer incidence, pancreatitis, retinopathy, cardiopathy, encephalopathy, peripheral neuropathy and renopathy; and (ii) administering systemically, a therapeutic amount of one or more formulations, of a synthesized curcumin formulation, wherein the formulations comprise a polylactic glycolic acid (PLGA) copolymer enveloped curcumin designed to be released over a ten day period, or a polymer conjugated nanocurcumin.

The liposomal curcumin formulation is typically administered in combination with a calcium channel blocker, antihistamines and a corticosteroid to prevent hemolysis and hypersensitivity reactions to the liposome in a sustained and specific manner. In one aspect the one or more proliferative diseases encountered with incretin mimetics and other anti-diabetic drugs include cancer of the pancreas, thyroid, and other tissues. An increase in pancreatitis has also been reported with different incretin mimetics. In another aspect the PLGA-curcumin is injected subcutaneously every 10 days. The other two formulations may be infused over one hour in administration schedules ranging from once weekly, to three times a week for control of blood sugar and disease or drug sequelae in combination with any intravenous, or subcutaneous, or oral anti-diabetic medication or combination of medications (example: sidagliptin and metformin).

The method as described hereinabove further comprises the step of adding curcumin formulations to diabetic drugs for treating systemic diabetes morbidity in humans. The method comprises administering a pharmaceutical composition intravenously or subcutaneously as a sustained release during repetitive 10 day cycles, which may be repeated depending upon tolerance, and therapeutic need. By combining these curcumin formulations with conventional anti-diabetic drugs, the adverse side effects of the drugs and the natural diabetes-related disease pathologies are mitigated. The compositions for use with the methods of the present invention include different formulations that enclose an effective amount of curcumin, increase aqueous solubility, enhance delivery to pathologic tissues, exert anti-hyperglycemic control and with the PLGA formulation administered subcutaneously, a slow infusion rate which allows continuous glycemic control along with oral or subcutaneous incretin mimetics for example, and enhances patient compliance, safety, and general health.

Pancreatic islet cell pathology: In type 2 diabetes the causes for beta cell insufficiency to secrete the required amount of insulin to maintain blood glucose at normal levels are not entirely clear. However, pancreatic islet pathology is comparable to neuropathic diseases in accumulation of protein islet amyloid polypeptide (IAPP) toxic oligomers. Islet cell amyloid is a normal constituent of beta cells and co-secreted with insulin in animals and man (Clark A 1996). Amyloid deposits are found in pancreatic islets of 90% of type 2 diabetic subjects at postmortem suggesting that progressive amyloidosis leads to islet secretary dysfunction (Jaikaran ET, 2001). Conversion from the IAPP soluble monomer, to beta-sheet fibrils involves changes in molecular configuration, cellular biochemistry, and other diabetes related factors. Extensive fibril invaginations in the β cell plasma membrane interfere with membrane signaling and insulin release exacerbating diabetes-associated islet dysfunction. When misfolded, amylin, a 37-amino acid polypeptide is secreted from pancreatic islet cells and converted to amyloid deposits. Extracellular deposition of amylin between beta cells and islet capillaries predominates in animal models and man. Eventually the islet cells are replaced by fibril formations (Clark A, 1996). Low micromolar levels of curcumin reduce polypeptide fibril formation and aggregates. Curcumin also partially protects beta INS-1 cells from exogenous islet amyloid polypeptide toxicity. Greater levels of curcumin may be cytotoxic and do not protect misfolded polypeptide overexpressing cells (Daval M, 2010).

Curcumin molecular effects in patients with type 2 diabetes: Curcumin does not have a direct effect on receptor tyrosine kinase, 2-deoxyglucose uptake in L6-GLUT4 myc cells, or intestinal glucose metabolism as measured by DPP-4/alpha-glucosidase inhibitory activity. Curcumin suppressed dexamethasone induced phosphoenol pyruvate carboxy kinase/alpha-glucosidase inhibitory activity (PEPCK), and glucose6-phosphotase (G6Pase) in H411E rat hepatoma and Hep3B human hepatoma cells. Curcumin also increases phosphorylation of AMP-activated protein kinase (AMPK) and its downstream target acetyl-CoA carboxylase (ACC) in H4IIE and Hep3B cells with 400 times the potency of metformin. The AMPK mediated suppression of hepatic gluconeogenesis is a mechanism mediating glucose-lowering effects of curcumin. (Kim T, 2009). Curcumin increases glucose kinase in the liver that converts glucose to glycogen stores hence lowering circulating glucose. Obesity associated with low grade tissue inflammation leads to activated macrophages in fat tissues which release the cytokine JNK-1 which causes cells to become insulin resistant: in this respect curcumin inhibits JNK-1 and increases insulin sensitivity.

Curcumin adverse effects: The insulin sensitizing thiazolidinediones, rosiglitazone and pioglitazone are peroxisome proliferator-activated receptor-γ (PPAR-γ agonists). Agonist activity has an immune-modulatory effect with an increased risk of pneumonia or lower respiratory tract infection with long-term use (Singh S, 2011). Since curcumin may also acts as a PPAR-γ agonist (Jacob A, 2007), it may exacerbate this risk and contraindicate its combination with either of these two drugs over extended periods of time. Alternatively, this may not be a contraindication since more recent data demonstrate that curcumin is not a PPAR-γ ligand (Narala VR 2009). Curcumin also decreases neutrophil migration and myeloperoxidase release indicating a reduction in neutrophil activation. On the other hand, curcumin enhances wound healing, and its anti-inflammatory activity appears to have a beneficial effect in sepsis. The beneficial anti-inflammatory effects of curcumin are mediated by the up-regulation of PPAR-γ regulation (Jacob A, 2007). The thiozolidinediones also have an associated increased risk of myocardial infarction and heart failure in patients with type-2 diabetes, however, without a significantly increased risk of cardiovascular mortality (Singe S, 2007).

DPP-4 blockade effects on type-2 diabetes: Oral DPP-4 inhibitors are used extensively for glycemic control. DPP-4 inhibition: lowers glucosylated hemoglobin (HBA1c), fasting and post-prandial glucose levels, stimulate insulin secretion in the presence of hyperglycemia, and inhibit glucagon secretion. Des-fluoro-gliptin in food achieves optimal glucagon like Peptide-1 (GLP-1) control by a potent and sustained 24-hour inhibition of DPP-4 activity (Lamont 2008). The DPP-4 blockers used as monotherapy however, may not offer adequate insulin/glucose control. Sitagliptin/metformin achieves greater improvements in glycemic control than either component alone (Chwieduk CM, 2011). The addition of curcumin formulations to this combination has not been explored.

DPP-4 inhibition and adverse events: There is an increased incidence and risk of pancreatitis, pancreatic, thyroid and other cancers among patients given GLP-1 based therapy (Elashoff M, 2011). These are mentioned in the clinical brochure accompanying the commercial GLP-1 product Sitagliptin. The incidence may be less than 1% since these are not mentioned in a review of specific clinical adverse events by system organ class of a pooled Phase II, III Sitagliptin treated population (2786 patients compared with 2355 non-exposed patients). This latter review of adverse events revealed that the event rate of skin and subcutaneous tissue disorders (contact dermatitis, urticaria) was increased. Sitagliptin treatment was also, associated with an increased incidence in bronchitis, nasal congestion, nasopharyngitis, tooth abscess, infestations, gastrointestinal disorders (upper abdominal pain, dyspepsia, gastritis), musculoskeletal disorders (myalgia, myopathy muscle weakness, meniscus lesions, osteoarthritis), and nervous system adverse events (tremors, balance disorders, ataxia, higher incidence of suicide ideation/completed suicide).

Pancreatitis: As early as 2002, it was reported that glyburide which stimulates the pancreas to produce insulin was associated with an increased risk for pancreatitis (Blomgren KB, 2002). In 2007, post-marketing reports of pancreatitis raised issues of a causal effect of exenatide, a glucagon-like peptide receptor agonist: two of six cases of hemorrhagic pancreatitis were lethal. Between June 2005 and 2008, 0.13 and 0.12% of 27,996 injectible exenatide and 16,276 oral sidagliptin users suffered acute pancreatitis. Two of 88 cases of acute pancreatitis reported with sitagliptin were hemorrhagic. The Incidence of pancreatitis in metformin-glyburide users is similar suggesting that a specific causal relation for incretin-mimetics is unlikely. This suggests that pancreatitis is constituitively increased in patients with type 2 diabetes, or promoted by these treatments (Olansky L, 2010). This is suggested by the observation that GLP-1 receptor activation and signaling increases pancreatic mass and modulates expression of genes associated with pancreatitis. It may be species specific since gliptin agonists do not modify the severity of experimental pancreatitis in mice (Koehler JA, 2009).

When pancreatitis develops in type-2 diabetic patients, the high morbidity and mortality is associated with up-regulation of a number of pro-inflammatory signaling molecules including AP-1, NF-kB, TNFα, IL-6, and IL-8: all of which lead to damaged pancreatic tissues. By blocking key signals of the inflammatory response in rats, curcumin inhibits cerulean (an analog of cholecystokinin), or an ethanol diet and low dose cholecystokinin induced pancreatitis. Pancreatitis indices in these animals included histology, serum lipase, amylase, pancreatic trypsin activation, neutrophil infiltration and inducible nitric oxide synthase. Curcumin's mechanisms of action include antioxidant effects, blockade of CCK-induced NF-kB and AP-1 in isolated pancreatic acini and attenuation of expression of NF-kb and AP-1, TNFα, IL6, and IL8 (Guvkovsky I, 2003).

Cancer: There is increased risk of cancer when using DPP-4 inhibitors due to down regulation of the tumor-suppressive activity of DPP-4. The cell surface protease DPP-4 limits tissue invasion of tumors, and is involved in peptide-mediated cell growth and differentiation. Its expression is lost in different histologic sub-types of human NSCLC cancer cells at both mRNA and protein levels (Pro B, 2004, Wesley UV, 2004 and 2005). Most insulin mimetics such as Sitagliptin are given in combination or as add-ons to Metformin. Metformin alone has potential antitumorigenic effects independent of its hypoglycemic effects. Purported mechanisms of action include activation of the LKB1/AMPK pathway, induction of cell cycle arrest and apoptosis, protein synthesis inhibition, reduced circulating insulin levels, inhibition of the unfolded protein response, activation of the immune system, and eradication of cancer stem cells (Kourelis TV, 2011). Curcumin has demonstrated anticancer activity (Bisht S 2010, Lim KJ, 2011). It may be particularly valuable against type 2 diabetes-associated pancreatic cancers when administered concomitantly with metformin. Curcumin also modulates angiogenesis where uncontrolled angiogenesis is associated with tumor growth and metastases. Expression of PPARδ is elevated in human and rat colorectal cells, and is implicated in growth of hepatocellular, cholangiocarcinoma, breast, prostate and other human cancers. Curcumin can inhibit the expression of PPARδ and related genes inducing down-regulation 14-3-3 epsilon and VEGF (Wang JB 2009).

Diabetic encephalopathy: In streptozotocin treated rats there are degenerative changes in neurons and glia, perivascular and mitochondrial swelling, myelin sheath disarrangements, increased areas of myelinated axons, presynaptic vesicle dispersion in swollen axonal boutons, neurofilament fragmentation, oligodendrocyte abnormalities, cell death, and behavioral depressive moods (Hernandez-Fonseca JP, 2009). In elderly human subjects with Type-2 diabetes, neuronal damage may be caused by hyperglycemia and excess intracellular glucose leading to oxidative stress and activation of signaling pathways altering gene expression and extracellular matrix (ECM) proteins (Brownlee M, 2001, Chen S, 2003). Gradually developing end-organ cerebral encephalopathy, retinopathy and nephropathy occurs with uncontrolled glucose levels. Diabetic encephalopathy is characterized by EEG, cell structural, dendritic pathology, chronic metabolic and vascular changes associated with mild to moderate cognitive impairment and increased risk of dementia. Type-2 diabetes increases the risk of dementia more than two fold if not controlled. Encephalopathy is characterized by reduced glutathione and superoxide dismutase, increased nitrite levels, 80% decreased cholinergic function in the cerebral cortex, 107% increase in thiobarbituric acid reactive substances in the cerebral cortex and 121% in the hippocampus. Serum TNFα may be increased 1100%. Reactive glial cells: a hallmark of neurodegenerative diseases release cytokines, reactive oxygen intermediates, nitric oxide, quinolinic acids, neurotoxins and glutamate (Kuhad A, 2007). Treatment with metformin and sulfonylureas were reported to decrease risk by 35% over eight years of study in 800,000 patients (Hsu CC, 2011). Incretin mimetics such as Sitagliptin have possible therapeutic activity in diabetic encephalopathy. The endogenous peptide hormone GLP-1 has cAMP coupled receptors within the brain of rodents and humans where it has a role in regulation of proper neuronal functioning and acts against excitotoxic induced cell death and oxidative injury (Perry T, 2002). GLP-1 reduces endogenous levels of amyloid β Peptide in mouse brain, and β amyloid precursor protein in neurons (Perry T, 2003). Mice gavaged with 20 mg/kg Sitagliptin for 12 weeks had increased levels of incretin GLP-1 in the brain. Amyloid β levels and amyloid plaques decreased about 50% and were associated with substantial reduction in cerebral inflammation and nitrosative stress. Decrease in GLP-1 with extendin (9-39), a specific potent glucagon-like peptide-1 receptor antagonist does not alter IAPP or β amyloid depletion in mice. Incretin (GLP-1) in brain tissue can be assayed to 2 μm sensitivity using a commercially available Elisa kit (Linco Research, St. Charles, Missouri).

Neuroprotective mechanisms of action of curcumin: Glutamate excitotoxicity is mediated by intracellular Ca(2+) overload, caspase 3 activation and ROS generation, all inhibited by curcumin. Curcumin inhibits PKC activity and subsequent phosphorylation of NR1 of the NMDA receptor, thereby reducing glutamate-mediated Ca(2+) influx. Curcumin also inhibited glutamate induced caspase-3 activation (Sugimoto H 2006).

Excess glutamate induces activation of c-Jun-terminal kinase (JNK) and p38 kinase to phosphorylate c-jun and increases activator protein-1 (AP-1) binding, which leads to cerebellar granule cell death. Exposure of rat cortical neurons to 10 mM glutamate for 24 hours decreased brain derived neurotrophic factor (BDNF) levels, reduced cell viability, and enhanced cell apoptosis. Pre-treatment with curcumin reversed these effects in a dose and time dependent manner. The curcumin survival-promoting effect can be blocked by K252a, a tyrosine receptor kinase (Trk) inhibitor, which inhibits activity of BDNF and suppresses curcumin up-regulation of BDNF. This indicated that the neuroprotective effect of curcumin is mediated by BDNF and the Trk signaling pathway (Wang R, 2008). Curcumin indirectly inhibits JNK by its action as an AP-1 binding inhibitor, hence has a neuroprotective effect against cerebral glutamate toxicity.

TNFα is a pro-inflammatory cytokine implicated in neuronal cell death. Chronic expression of high levels can lead to progressive neuronal loss, gliosis, and inflammatory infiltrates of monocytes and macrophages. Fetal brain cell cultures exposed to excitatory glutamate for 6 days undergo a dose dependent cell loss potentiated by TNFα, which directly affects glutamate metabolism (Chao CC, 1994). Curcumin can prevent high levels of TNFα if administered chronically, and lowered levels can be protective via mediation of THFR-1, GDNF, and SOD (Chertoff M, 2011).

Glutathione plays an important role in neuroprotection from neurotoxicity by modulating brain thiol status. Free radical production is increased in subjects with type 2 diabetes. But the mechanism responsible for the linkage among increased oxidative stress and impaired glucose metabolism is not clearly defined. Intracellularly, glutathione has anti-oxidant properties inhibiting free-radical formation and functions as a redox buffer (Collier A, 1990). Glutathione peroxidase-1 contributes to the neuroprotection in ischemia—reperfusion injury (Crack P J, 2003). Alternatively, some chemicals can be converted to neurotoxicants following conjugation with glutathione.

Neuroregeneration is part of the neuro-protective mechanisms of action. In this respect curcumin has several positive attributes. The formulated compound passes the blood brain barrier and localizes in the hippocampus (Chiu S, 2011, Ray B 2010). In in vitro studies, hippocampus cells were stimulated to grow and exhibited neuroplasticity (Kim S J, 2008). On a molecular basis curcumin prevents neurotoxicity by suppressing cytokine release from activated microglia, glutamate induced, excitotoxicity, up regulation of the transcription factor nuclear factor erythroid-2 related factor (Nrf2) which coordinates gene expression required for free radical scavenging, detoxification of xenobiotics and maintenance of redox potential (Yang 2009). Curcumin promotes the viability of cultured rodent cortical neurons by increasing BDNF and phosphor-TrkB which can be blocked by inhibiting the activity of BDNF with tyrosine kinase B antibody, or by the inhibitors of ERK, and PI3K (Wang 2010). Curcumin also induces increases in phosphorylated cAMP response-element binding protein (CREB), which can be prevented by PI3K and MAOK inhibitors. These observations suggest that curcumin neuroprotection is mediated via a signaling pathway including BNDF/TrkB-MAPK/PI3K-CREB. Curcumin also has additional activity in stimulating BDNF and its downstream effectors on synaptic plasticity (CREB, synapsin1) and CAMK II neuronal signaling (Wu A 2011).

Nitrite lowering; nitrite levels are significantly elevated in the cerebral cortex (112%) and hippocampus (94%) of streptozotocin treated rats. Curcumin treatment significantly lowers this increase in different regions without affecting total brain nitrite levels (Kuhad A, 2007). There are data indicating that Sitagliptin induces a reduction of inflammation and nitrosative stress within the brain in association with reduced amyloid deposition in a dose related manner (D'Amico M, 2009).

Hence, both agents curcumin and glyptin agonists combined may have additive or synergistic beneficial effects. Dopaminergic receptors are super sensitive to stress and glutamate excite-toxicity, and this may be modulated by curcumin's anti-inflammatory-anti-oxidant activity against induced stress, CREB, and phospholipase C (Kumar T P, 2010).

Stem-cell generation and differentiation effects of curcumin: Curcumin not only prevents death in animal models of neurodegenerative disorders, but in low concentrations (0.2 mg/kg IP or 4-5 μg in mice) activates extracellular-signal related kinases (ERK) and p38 kinases in multi-potent adult neural progenitor cells and adult hippocampal neurons. Inducing neurogenesis and neuroplasticity. Higher concentrations are cytotoxic (Kim S, 2008). Endogenous pancreatic adult stem cell neogenesis and differentiation is putatively associated with gastrin and TGF-α (Wang TC, 1993). Since the TGFα receptor interacts similarly to the EGF receptor and curcumin can blockade the EGF receptor, there is little probability that curcumin will contribute to pancreatic stem cell neogenesis.

Diabetic retinopathy: Proliferative diabetic retinopathy, a common cause of visual impairment is characterized by the growth of abnormal blood vessels in the back of the eye which leak blood into the center of the eyes causing retinal damage. After 10 years of disease duration, 75% of subjects will have damaged vision. Reduction in the rate of progression requires intensive glycemic control and treatment of dyslipidemia. Oxidative stress and inflammation implicated in the pathogenesis of diabetic retinopathy may be prevented by curcumin. Treatment with curcumin induces hypoglycemic activity, positively modulates the retinal antioxidants glutathione, superoxide dismutase and catalase and prevents elevated levels of tumor necrosis factor α, vascular endothelial growth factor, degeneration of endothelial cell organelles and increases in capillary basement membrane thickness of the retina (Gupta SK, 2011). The anti-angiogenic effects of curcumin contribute to mitigating progression of retinopathy, and inhibition of basic fibroblast-growth factor induced corneal neovascularization in the mouse cornea (Arbiser J L, 1998). Curcumin prevents diabetes-induced decrease in anti-oxidant capacity and elevation of IL-1b, VEGF, and NF-kB in Streptozotocin treated rats (Kowluru R A, 2007).

Diabetic nephropathy: This is the commonest cause of end-stage renal disease. Diabetic nephropathy secondary to hyperglycemia and associated increased oxidative stress activating signaling pathways and gene expression is characterized by thickening of the glomerular basement membrane and mesangial matrix expansion due to increased production of extracellular matrix proteins (Schena FP, 2005).

Endothelin-1, mediated expression of extracellular matrix proteins, fibronectin and a splice variant extradomain-B-containing fibronectin (EDB+FN) are common in the kidney and in other organs in patients with chronic diabetes. In patients with diabetic nephropathy EDB+FN, is increased in the serum. These changes are associated with TGF-β1 and NF-kB, both suppressed by curcumin. Activation of the nuclear hormone receptor peroxisome proliferator activated receptor (PPAR)-6 improves insulin resistance, adiposity and plasma HDL levels and is reno-protective in streptozotocin induced diabetic nephropathy (Matsushita Y, 2011). Short-term treatment of diabetic rats with curcumin prevents diabetes induced decreased anti-oxidant levels and kidney dysfunction (Chiu J, 2009).

To facilitate the understanding of the invention it is necessary to identify the molecular physiopathology in the multiple tissues affected by the diabetic syndrome, and to match those with the beneficial effects of curcumin. The total body of evidence presented of both a large variety of diabetic induced pathogenic changes, anti-diabetic drug effects and curcumin's mitigating multitargeted activities strongly support the combined use of curcumin to other drugs used in type 2 diabetic patients.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB.

Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

1. U.S. Patent Application No. 20100239552: Combination Therapies for Treating Metabolic Disorders.
2. WIPO Patent Application No. WO/2004/047717: Topical Application of Curcumin for the Treatment of Peripheral Neuropathy.
3. U.S. Patent Application No. 20100240581: Selective Proteasome Inhibitors for Treating Diabetes.
4. U.S. Patent Application No. 20100179103: Curcumin Cyclodextrin Combination for Preventing or Treating Various Diseases.
5. Anderson S L, Trujillo J M: Association of pancreatitis with glucagon-like peptide-1 agonist use. Ann Pharmacother. 2010, 44(5) 904-909.
6. Arbiser J L, Klauber N, Rohan R, et al.: Curcumin is an in vivo inhibitor of angiogenesis. Molecular Medicine 1998, 4(6):376-383.
7. Bisht S, Mizuma M, Feldman G et al: Systemic administration of polymeric nanoparticle-encapsulated curcumin(Nanocurc) blocks tumor growth and metastases in preclinical models of pancreatic cancer. Mol Cancer Ther 2010, 9: 2255-2264.
8. Blomgren K B, Steineck G, Sundstrom A, Wiholm B E: Obesity and treatment of diabetes with glyburide may both be risk factors for acute pancreatitis. Diabetes Care. 2002, 25(2):298-302.
9. Brownlee M.: Biochemistry and Molecular Cell Biology of diabetic complications. 2001, Nature 414: 813-820.
10. Chao C C, Hu S, Peterson P K: Glia: the not so innocent bystanders. J. Neurovirology. 1994, 2(4): 234-239.
11. Chen S, Mukherjee S, Chakraborty C, Chakrabarti S: High glucose induced, endothelin-dependent fibronectin synthesis is mediated by NF-kappaB and AP-1. Am J Physiol Cell Physiol. 2003, 284:C263-272.
12. Chertoff M, Di Paolo N, Schoeneberg A, et al; Neuroprotective and neurodegenerative effects of the chronic expression of tumor necrosis factor in the nigrostriatal dopaminergic circuit of adult mice. 2011, 227(2): 237-251.
13. Chiu J, Kahn Z A, Farhangkhoee H, Chakrabarti S: Curcumen prevents diabetes-associated abnormalities in the kidneys by inhibiting p300 and nuclear factor NF— B. Nutrition 2009, 25: 964-972.
14. Chiu S, Lui E. Majeed M et al: Curcumin Distribution in the Rat Brain. Anticancer Res 2011, In Press.
15. Chwieduk C M: Sitagliptin/Metformin fixed-dose combination: in patients with type 2 diabetes mellitus. Drugs 2011, 71(3) 349-361.
16. Clark A, Charge, S B, Badman M K, de Koning E J: APMIS, 1996 104(1): 12-18.
17. Collier A, Wilson R, Bradley H, Thompson J A, Small M: Free radical activity in type II diabetes, Diab Med 1990, 7:27-30.
18. Crack P J, Tayler J M, de Haan J B, Kola I, Hertzog P, Ianello R C: Glutathione peroxidase-1 contributes to the neuroprotection seen in the superoxide dismutase-1 transgenic mouse in response to ischemia/reperfusion injury. J Cereb Blood Flow Metab 2003, 23(1): 19-22.
19. D'Amico M, Di Fillipo C, Marfella R et al.: Long-term inhibition of dipeptidyl peptidase-4 in Alzheimer's prone mice. Experimental Gerontology 2010, 45:202-207.
20. Daval M, Bedrood S, Gurio T et al: The effect of curcumin on human islet amyloid polypeptide misfolding and toxicity. Amyloid 2010, 17(3-4): 118-128.
21. Elashoff M, Matveyenko A V, Gier B, Elashoff R, Butler P C: Gastroenterology 2011 Feb. 17, [Epub ahead of print].
22. Flint A, Kapitza C, Hindsberger C, Zdravkivic M: The once daily human glucagon-like peptide-1(GLP-1) analog liraglutide improves postprandial glucose levels in type 2 diabetes patients. Adv Ther 2011 Feb. 17 [Epub ahead of print].
23. Gupta S K, Kumar B, Nag T C et al: Curcumin Prevents Experimental Diabetic Retinopathy in Rats through its Hypoglycemic, Antioxidant and anti-inflammatory Mechanisms. J Ocul Pharmacol Ther 2011 Feb. 12 [Epub ahead of print]
24. Guvkovsky I, Reyes C N, Vaqero E C, Gukovskaya A S Pandol S J: Curcumin ameliorates ethanol and non ethanol experimental pancreatitis. Am J Physiol Gastrointest Liver Physiol. 2003, 284: G85-G95.
25. Hernandez-Fonseca J P, Rincon J, Pedreanez A, et al: Structural and ultrastructural analysis of cerebral cortex, cerebellum, and hypothalamus from diabetic rats. Exp Diabetes Res 2009 Oct. 1: 2009: 329632. [Epub ahead of print].
26. Hsu C C, Wahlqvist M L, Lee M S, Tsai H N: Incidence of Dementia is increased in Type 2 Diabetes and Reduced by the Use of Sulfonylureas and Metformin J Alzheimer's Dis. 2011, [Epub ahead of print].
27. Jacob A, Wu R, Zouh M, and P Wang: Mechanism of the anti-inflammatory effect of curcumin: PPAR-γActivation. PPAR Research 2007, 2007:89369.
28. Jaikaran E T, Clark A: Islet amyloid and type 2 diabetes: from molecular misfolding to islet pathophysiology. Biochim Biophys Acta 2001, 1537(3): 179-203.
29. Kim S J, Son T G, Park H R, et al.: Curcumin Stimulates Proliferation of Embryonic Neural Progenitor cells and Neurogenesis in the Adult Hippocampus. 2008 J Biol Chem, 283(21): 14497-14505.
30. Kim T, Davis J, Zhang A J, He X, Mathews S T: Curcumin activates AMPK and suppresses gluconeogenic gene expression in hepatoma cells. Biochem Biophys Res Comm 2009, 388(2): 377-382.
31. Koehler J A, Baggio L L, Lamont B J, Ali S, Drucker D J: Glucagon-like peptide-1 receptor activation modulates pancreatitis-associated gene expression but does not modify the susceptibility to experimental pancreatitis in mice. Diabetes 2009, 58(9): 2148-2161.
32. Kourelis T V, Siegel R D: Metformin and cancer: new applications for an old drug. Med Oncol 2011, Feb. 8 [Epub ahead of print].
33. Kowluru R A and Kanwar M: Effects of curcumin on retinal oxidative stress and inflammation in diabetes. Nutrition and Metabolism 2007, 4:8.
34. Kuhad A and Chopra K: Curcumin attenuates diabetic encephalopathy in rats: behavioral and biochemical evidences. European J. Pharmacology. 2007, 576: 34-42.
35. Kumar T P, Antony S, Gireesh G, George N, Paulose C S: Curcumin modulates dopaminergic receptor, CREB and Phospholipase c gene expression in the cerebral cortex and cerebellum of streptozotocin induced diabetic rats. 2010, J Biomed Sci. 17:43.
36. Lamont B J, and Drucker D J: Differential anti-diabetic efficacy of incretin agonists vs DPP-4 inhibition in high fat fed mice Diabetes. 2008, 57:190-198.
37. Li X, Lu F, Tian Q, Yang Y, Wang Q, Wang JZ: Activation of glycogen synthase kinase-3 induces Alzheimer-like tau hyperphosphorylation in rat hippocampus slices in culture. J Neurol Transm 2006, 113(1): 93-102.
38. Lim K J, Bisht S, Bar E E, Maitra, A, Eberhart C G: A polymeric nanoparticle formulation of curcumin inhibits growth, clonogenicity and stem-like fraction in malignant brain tumors. Cancer Biology and therapy 2011, 11:464-473.
39. Matsushita Y, Ogawa D, Wada J et al.: Activation of peroxisome proliferator-activated receptor 6 inhibits streptozotocin induced diabetic nephropathy through anti-inflammatory mechanisms in mice. Diabetes 2011, 60(3) 960-968.
40. Narala V R, Smith M R, Adapala R K et al.: Curcumin is not a ligand for peroxisome proliferator-activated receptor-gamma. Gene Ther Mol Biol 2009, 13(1):20-25.
41. Olansky L: Do incretin-based therapies cause acute pancreatitis? J Diabetes Sci Technol 2010, 4(1): 228-229.
42. Perry T and Greig N H: The glucagon like peptides: a new genre in therapeutic targets for intervention in Alzheimer's disease. J Alzheimer's Dis 2002, 4:487-496.
43. Perry T, Lahiri D R, Sambamurti K et al.: Glucagon-like peptide-ldecreases endogenous amyloid-beta peptide levels and protects hippocampal neurons from death induced by A beta and iron. 2003, J Neuroscience Res 72 603-612.
44. Pfutzner A, Paz-Pacheko E, Allen E M Frederich B, Chen R: Initial combination therapy with saxagliptin and metformin provides sustained glycaemic control and is well tolerated for up to 76 weeks. Diabetes Obes Metab 2011, Feb. 22 [Epub ahead of print].
45. Pro B, Dang N H: “C26/dipeptidyl peptidaseIV and its role in cancer. Histol Histopathol 2004, 19(4): 1345-1351.
46. Ray B, Bisht S, Maitra Am, Maitra A, Lahiri D K: Neuroprotective and neurorescue effects of a novel polymeric nanoparticle formulation of curcumin(Nanocurc) in the neuronal cell culture and animal model:implications for Alzheimer's disease. J Alzheimer's disease 2011, 23:61-77.
47. Roberts A N, Leighton B, Todd J A et al: Molecular and functional characterization of amylin, a peptide associated with type 2 diabetes mellitis. Proc Natl Acad Sci USA 1989, 86(24): 9662-9666.
48. Schena F P, Gesualdo L: Pathogenetic mechanisms of diabetic nephropathy. J Am Soc Nephrol 2005, 16 (suppl 1): S30-33.
49. Singh S, Loke Y K, Furberg C D: Long-term risk of cardiovascular events with rosiglitazone:a meta-analysis. JAMA 2007, 298(10): 1189-1195.
50. Singh S, Loke Y K, Furberg C D: Long-term use of thiazolidinedions and the associated risk of pneumonia or lower respiratory tract infection: systematic review and meta-analysis. Thorax 2011, Feb. 15 [Epub ahead of print).
51. Sugimoto H, Shen H, Yazawa K, Kihara T, Nidome T, Shimmyo Y: Curcumin and tannic acid protect against glutamate-induced excitotoxicity. FEBS Lett 2006, 580(28-29):6623-6628.
52. Wang J B, Qi L L, Zheng S D, Wang H Z, Wu T X: Curcumin suppresses PPAR expression and related genes in HT-29 cells. World J Gastroenterol 2009, 15(11): 1346-1352.
53. Wang R, Li Y H, Xu Y et al.: Curcumin produces neuroprotective effects via activating brain derived neurotrophic factor/TrkB-dependent MAPK and PI3K cascades in rodent cortical neurons. Prog Neuropsychopharmacol Biol Psychiatry. 2010, 34:147-153.
54. Wang T C, Bonner-Weir S, Oates P S et al.: Pancreas gastrin stimulates islet differentiation of transforming growth Factor α-induced ductular precursor cells. J Clin Inv 1993, 92:1349-1356.
55. Wesley U V, Tiwari T, Houghton A N: Role for dipeptidyl peptidase IV in tumor suppression of human non small cell lung carcinoma cells. Int J Cancer 2004, 109(6): 855-866.
56. Wesley U V, McGroarty M, HomoyouniA: Dipeptidyl peptidase inhibits malignant phenotype of prostate cancer cells by blocking basic fibroblast growth factor signaling pathway. Cancer Res 2005, 65(4): 1325-1334.
57. Wu A, Ying Z, Schubert D, Gomez-Pinella F: Brain and spinal cord interaction: A dietary curcumin derivative counteracts locomoter and cognitive deficits after brain trauma. Neurorehabil Neural Repair 2011, Feb. 22 [Epub ahead of print].
58. Yang C, Zhang X, Fan H, Liu Y: Curcumin up-regulates transcription factor Nrf2, HO-1 expression and protects rat brains against focal ischemia. Brain Res 2009, 1282:133-141.

What is claimed is:

1. A composition for ameliorating symptoms and/or treating type 2 diabetes and one or more associated pathological conditions (sequelae) in a human subject comprising:
a therapeutically effective amount of a formulation comprising curcumin, curcumin analogues, curcumin derivatives, curcuminoids or combinations thereof dissolved or dispersed in a suitable aqueous or non-aqueous medium, wherein the curcumin is enclosed in at least one of one or more liposomes, enclosed in a polymeric nanoparticle, conjugated to one or more biodegradable polymers or formulated into a liposome that is surrounded or incorporated into a polymeric nanoparticle;
a therapeutically effective amount of one or more anti-diabetic agents; and
one or more optional excipients, diluents, extended or controlled release agents, lubricants, preservatives or any combinations thereof.

2. The composition of claim 1, wherein the formulation is administered concomitantly with the one or more anti-diabetic agents, wherein the anti-diabetic agents may be administered orally, intravenously, or subcutaneously.

3. The composition of claim 1, wherein the type 2 diabetes associated pathological condition is at least one of cerebral, cardiac, pancreatic, renal, an ocular condition, pancreatitis, retinopathy, cardiopathy, encephalopathy, peripheral neuropathy and renopathy, or increased cancer incidence.

4. The composition of claim 1, wherein the one or more biodegradable polymers are selected from the group consisting of polyesters, polylactides, polyglycolides, polycaprolactones, polyanhydrides, polyamides, polyurethanes, polyesteramides, polydioxanones, polyacetals, polyketals, polycarbonates, polyorthocarbonates, polyorthoesters, polyphosphoesters, polyphosphazenes, polyhydroxybutyrates, polyhydroxyvalerates, polyalkylene oxalates, polyalkylene succinates, poly(malic acid), poly(amino acids), copolymers, poly-lactic glycolic acid (PLGA) copolymer, terpolymers, and combinations or mixtures thereof.

5. The composition of claim 1, wherein the PLGA copolymer enclosed curcumin releases 50% of the curcumin within the first 24 hours and 20-50% of the remaining curcumin within 2-10 days following a subcutaneous injection.

6. The composition of claim 1, wherein the liposome comprises a lipid or a phospholipid wall, wherein the lipids or the phospholipids are selected from the group consisting of phosphatidylcholine (lecithin), lysolecithin, lysophosphatidylethanol-amine, phosphatidylserine, phosphatidylinositol, sphingomyelin, phosphatidylethanolamine (cephalin), cardiolipin, phosphatidic acid, cerebrosides, dicetylphosphate, phosphatidylcholine, and dipalmitoyl-phosphatidylglycerol, stearylamine, dodecylamine, hexadecyl-amine, acetyl palmitate, glycerol ricinoleate, hexadecyl sterate, isopropyl myristate, amphoteric acrylic polymers, fatty acid, fatty acid amides, cholesterol, cholesterol ester, diacylglycerol, and diacylglycerolsuccinate.

7. The composition of claim 1, wherein the anti-diabetic agent comprises insulin, pramlitide, bydureone, metformin, oral incretin mimetics, metformin, or any combinations thereof.

8. The composition of claim 1, wherein the formulation may be administered with one or more agents to prevent hemolysis, hypersensitivity reactions or both, wherein the agents comprise calcium channel blockers, antihistamines, corticosteroid or any combinations thereof.

9. The composition of claim 8, wherein the calcium channel blockers comprise verapamil, ethylisopropylameloride, niflamic acid, NPPB, dihydropyridines, phenylalkylamines, benzothiozepines, diltiazem, non-selective blockers comprising mibefradil, bepredil, fendeline, fluspirilene, catecholamines, and erythropoietin agents.

10. The composition of claim 1, wherein the nanoparticle comprises a copolymer comprising isopropylacrylamide, (NIPAAM), N-vinyl pyrrolidinone (VP) and acrylic acid (AA).

11. A method for ameliorating symptoms and/or treating type 2 diabetes and one or more associated pathological conditions (sequelae) in a human subject comprising the steps of:
identifying the human subject in need of amelioration of the symptoms and/or treatment of type 2 diabetes and one or more associated pathological conditions (sequelae); and
administering a therapeutically effective amount of a pharmaceutical composition to the human subject, wherein the composition comprises:
curcumin, curcumin analogues, curcumin derivatives, curcuminoids or combinations thereof dissolved or dispersed in a suitable aqueous or non-aqueous medium, wherein the curcumin is enclosed in at least one of one or more liposomes, enclosed in a polymeric nanoparticle, conjugated to one or more biodegradable polymers or formulated into a liposome that is surrounded or incorporated into a polymeric nanoparticle;
one or more anti-diabetic agents; and
one or more optional excipients, diluents, extended or controlled release agents, lubricants, preservatives or any combinations thereof.

12. The method of claim 11, wherein the formulation is administered concomitantly with the one or more anti-diabetic agents, wherein the anti-diabetic agents may be administered orally, intravenously, or subcutaneously.

13. The method of claim 11, wherein the type 2 diabetes associated pathological condition is a cerebral, cardiac, pancreatic, renal, or an ocular condition pancreatitis, retinopathy, cardiopathy, encephalopathy, peripheral neuropathy and renopathy, increased cancer incidence, or any combinations thereof.

14. The method of claim 11, wherein the one or more biodegradable polymers are selected from the group consisting of polyesters, polylactides, polyglycolides, polycaprolactones, polyanhydrides, polyamides, polyurethanes, polyesteramides, polydioxanones, polyacetals, polyketals, polycarbonates, polyorthocarbonates, polyorthoesters, polyphosphoesters, polyphosphazenes, polyhydroxybutyrates, polyhydroxyvalerates, polyalkylene oxalates, polyalkylene succinates, poly(malic acid), poly(amino acids), copolymers, poly-lactic glycolic acid (PLGA) copolymer, terpolymers, and combinations or mixtures thereof.

15. The method of claim 11, wherein the liposome comprises a lipid or a phospholipid wall, wherein the lipids or the phospholipids are selected from the group consisting of phosphatidylcholine (lecithin), lysolecithin, lysophosphatidylethanol-amine, phosphatidylserine, phosphatidylinositol, sphingomyelin, phosphatidylethanolamine (cephalin), cardiolipin, phosphatidic acid, cerebrosides, dicetylphosphate, phosphatidylcholine, and dipalmitoyl-phosphatidylglycerol, stearylamine, dodecylamine, hexadecyl-amine, acetyl palmitate, glycerol ricinoleate, hexadecyl sterate, isopropyl myristate, amphoteric acrylic polymers, fatty acid, fatty acid amides, cholesterol, cholesterol ester, diacylglycerol, and diacylglycerolsuccinate.

16. The method of claim 11, wherein the PLGA copolymer enclosed curcumin releases 50% of the curcumin within the first 24 hours and 20-50% of the remaining curcumin within 2-10 days following a subcutaneous injection.

17. The method of claim 11, wherein the anti-diabetic agent comprises insulin, pramlitide, bydureone, metformin, oral incretin mimetics, metformin, or any combinations thereof.

18. The method of claim 11, wherein the formulation may be administered with one or more agents to prevent hemolysis, hypersensitivity reactions or both, wherein the agents comprise calcium channel blockers, antihistamines, corticosteroid or any combinations thereof.

19. The method of claim 18, wherein the calcium channel blockers comprise verapamil, ethylisopropylameloride, niflamic acid, NPPB, dihydropyridines, phenylalkylamines, benzothiozepines, diltiazem, non-selective blockers comprising mibefradil, bepredil, fendeline, fluspirilene, catecholamines, and erythropoietin agents.

20. The method of claim 11, wherein the nanoparticle comprises a copolymer comprising isopropylacrylamide, (NIPAAM), N-vinyl pyrrolidinone (VP) and acrylic acid (AA).

21. A pharmaceutical composition for combination therapy to provide enhanced glycemic control in type 2 diabetes, prevent or treat one or more pathological conditions associated with type 2 diabetes, or both comprising:
curcumin, curcumin analogues, curcumin derivatives, curcuminoids or combinations thereof dissolved or dispersed in a suitable aqueous or non-aqueous medium, wherein the curcumin is enclosed in at least one of one or more liposomes, enclosed in a polymeric nanoparticle, conjugated to one or more biodegradable polymers or formulated into a liposome that is surrounded or incorporated into a polymeric nanoparticle; and
one or more anti-diabetic agents comprising DPP-4 inhibitors, insulin release sensitizers, glucagon modifiers or any combinations thereof.

22. The composition of claim 21, wherein the composition comprises Curcumin-DPP4-inhibitor-metformin.

23. The composition of claim 21, wherein the composition comprises Curcumin-bydureon-slow release-metformin.

24. The composition of claim 21, wherein the composition has fewer adverse reactions than DPP4-inhibitor-metformin, bydureon-slow release-metformin or both.

25. The composition of claim 21, wherein the composition provides enhanced glycemic control when compared to DPP4-inhibitor-metformin, bydureon-slow release-metformin or both.

26. A method of providing enhanced glycemic control in type 2 diabetes, preventing or treating one or more pathological conditions associated with type 2 diabetes, or both in a human subject comprising the steps of:
identifying the human subject in need of enhanced glycemic control in type 2 diabetes, prevention or treatment of one or more pathological conditions associated with type 2 diabetes, or both; and
administering a therapeutically effective amount of a pharmaceutical composition to the human subject comprising:
curcumin, curcumin analogues, curcumin derivatives, curcuminoids or combinations thereof dissolved or dispersed in a suitable aqueous or non-aqueous medium, wherein the curcumin is enclosed in at least one of one or more liposomes, enclosed in a polymeric nanoparticle, conjugated to one or more biodegradable polymers or formulated into a liposome that is surrounded or incorporated into a polymeric nanoparticle; and
one or more anti-diabetic agents comprising DPP-4 inhibitors, insulin release sensitizers, glucagon modifiers or any combinations thereof.

27. The method of claim 26, wherein the one or more pathological conditions associated with type 2 diabetes comprises encephalopathy, retinopathy, nephropathy, pancreatitis, pancreatic, cancer thyroid cancer, and other cancers.

28. The method of claim 26, wherein the composition may comprise one or more excipients, diluents, extended or controlled release agents, lubricants, preservatives or any combinations thereof.

29. The method of claim 26, wherein the composition comprises Curcumin-DPP4-inhibitor-metformin or Curcumin-bydureon-slow release-metformin.

30. A pharmaceutical composition for combination therapy to provide enhanced glycemic control in type 2 diabetes, prevent or treat one or more pathological conditions associated with type 2 diabetes, ameliorate one or more adverse reactions associated with type 2 diabetes treatment or any combinations thereof comprising:
curcumin, curcumin analogues, curcumin derivatives, curcuminoids or combinations thereof dissolved or dispersed in a suitable aqueous or non-aqueous medium, wherein the curcumin is enclosed in at least one of one or more liposomes, enclosed in a polymeric nanoparticle, conjugated to one or more biodegradable polymers or formulated into a liposome that is surrounded or incorporated into a polymeric nanoparticle; and
one or more anti-diabetic agents comprising DPP-4 inhibitors, insulin release sensitizers, glucagon modifiers or any combinations thereof.

31. A method of providing enhanced glycemic control in type 2 diabetes, preventing or treating one or more pathological conditions associated with type 2 diabetes, ameliorating adverse reactions associated with type 2 diabetes treatment or any combinations thereof in a human subject comprising the steps of:
identifying the human subject in need of enhanced glycemic control in type 2 diabetes, prevention or treatment of one or more pathological conditions associated with type 2 diabetes, amelioration or prevention of adverse reactions associated with type 2 diabetes treatment or any combinations thereof; and
administering a therapeutically effective amount of a pharmaceutical composition to the human subject comprising:
curcumin, curcumin analogues, curcumin derivatives, curcuminoids or combinations thereof dissolved or dispersed in a suitable aqueous or non-aqueous medium, wherein the curcumin is enclosed in at least one of one or more liposomes, enclosed in a polymeric nanoparticle, conjugated to one or more biodegradable polymers or formulated into a liposome that is surrounded or incorporated into a polymeric nanoparticle; and
one or more anti-diabetic agents comprising DPP-4 inhibitors, insulin release sensitizers, glucagon modifiers or any combinations thereof.

32. A composition for ameliorating symptoms and/or treating type 2 diabetes and one or more associated pathological conditions (sequelae) in a human subject comprising:
a therapeutically effective amount of a formulation comprising curcumin, curcumin analogues, curcumin derivatives, curcuminoids or combinations thereof enclosed in a liposome, and wherein the liposomes are enclosed in a polymeric nanoparticle, wherein the formulation inhibits a cardiac potassium channel blocking activity of curcumin; and
one or more optional excipients, diluents, extended or controlled release agents, lubricants, preservatives or any combinations thereof.

33. A method of providing enhanced glycemic control in type 2 diabetes, preventing or treating one or more pathological conditions associated with type 2 diabetes, ameliorating adverse reactions associated with type 2 diabetes treatment or any combinations thereof in a human subject comprising the steps of:
identifying the human subject in need of enhanced glycemic control in type 2 diabetes, prevention or treatment of one or more pathological conditions associated with type 2 diabetes, amelioration or prevention of adverse reactions associated with type 2 diabetes treatment or any combinations thereof; and
administering a therapeutically effective amount of a pharmaceutical composition to the human subject comprising:
a therapeutically effective amount of a formulation comprising curcumin, curcumin analogues, curcumin derivatives, curcuminoids or combinations thereof enclosed in a liposome, and wherein the liposomes are enclosed in a polymeric nanoparticle, wherein the formulation inhibits a cardiac potassium channel blocking activity of curcumin; and
one or more optional excipients, diluents, extended or controlled release agents, lubricants, preservatives or any combinations thereof.
